(19) United States
(12) Patent Application Publication
(10) Pub. No.: US 2006/0269826 A1
(43) Pub. Date: Nov. 30, 2006

(54) Novel electrode with switchable and tunable power output and fuel cell using such electrode

(76) Inventors: Eugenii Katz, Jerusalem (IL); Itamar Willner, Mevasseret Zion (IL)

Correspondence Address: NATH & ASSOCIATES, 112 South West Street, Alexandria, VA, 22314, US

(21) Appl. No.: 10/547,617

(22) PCT Filed: Mar. 2, 2004
(86) PCT No.: PCT/IL04/00199

§ 371 Date: Mar. 10, 2006

Related U.S. Application Data
(60) Provisional application No. 60/450,702, filed on Mar. 3, 2003.

(51) Int. Cl. H01M 4/90; G01N 33/487

(52) U.S. Cl. 429/42; 429/43; 204/403.01

(57) ABSTRACT

The present invention provides a novel electrode carrying on at least a portion of its support surface a hybrid polymer matrix (HPM), a catalyst that can catalyze a redox reaction and an optional electron mediator group that enhances the electrical contact between the HPM and the catalyst, the HPM being capable to be electrochemically changed from a non-conductive state to a conductive state. The electrode of the invention may be used in electrical devices such as fuel cells, thus imparting them switchable and tunable properties. The fuel cell of the invention may be used as a power source or as a self-powered sensor.

### FIELD OF THE INVENTION

The present invention is in the field of biocatalytic systems. More specifically, the present invention relates to biocatalytic electrodes and fuel cells capable of operation in a biological system and methods of their manufacture and use.

### LIST OF REFERENCES

In the following description reference will be made to several prior art documents shown in the list of references below. The reference will be made by indicating in brackets their number from the list.
(1) Willner, I.; Heleg-Shabtai, V.; Blonder, R.; Katz, E.; Tao, G.; Bückmann, A. F.; Heller, A. : Am. Chem. Soc. 1996, 118, 10321-10322.
(2) Katz, E.; Rildin, A.; Heleg-Shabtai, V.; Willner, I.; Bückmann, A. F. Anal. Chim. Acta 1999, 385, 45-58.
(3) Zayats, M.; Katz, E.; Willner, I. J Am. Chem. Soc. 2002, 124, 2120-2121.
(4) Raitman, O. A.; Patolsky, F.; Katz, E.; Willner, I. Chem. Commun. 2002, 1936-1937.
(5) Katz, E.; Willner, I.; Kotlyar, A. B. J Electroanal. Chem. 1999, 479, 64-68.
(6) WO 03/019170
(7) Chegel, V. I.; Raitman, O. A.; Lioubashevski, O.; Shirshov, Y.; Katz, E.; Willner, I. Adv. Mater. 2002, 14, 1549-1553.
(8) Gileadi, E.; Tsionsky, V. J. Electrochem. Soc. 2000, 147, 567-574.
(9) Morris, D. L.; Buckler, R. T. In: Methods in Enzymology; Langone, J. J., Van Vunakis, H., Eds.; Academic Press: Orlando, Fla. 1983; Vol. 92, Part E, pp. 413-417.
(10) Yonetani, T. J. Biol. Chem. 1961, 236, 1680.
(11) Katz, E.; De Lacey, A. L.; Fernandez, V. M. J Electroanal. Chem. 1993, 358, 261-272.

### BACKGROUND OF THE INVENTION

Electrical contacting of redox enzymes with electrode supports attracts substantial research efforts directed to the development of biosensors, bioelectrocatalyzed chemical transformations, and the development of biofuel cell elements. Tethering of electroactive relays to redox proteins or the immobilization of redox proteins in electroactive polymers are common practices to electrically contact and activate the redox enzymes.

The effective electrical contacting of redox-enzymes on electrodes by their structural alignment on electrodes through the surface reconstitution of flavoenzymes or pyrroloquinoline quinone (PQQ)-dependent enzymes on a relay-FAD monolayer assembly (1-3) or redox polymer-PQQ thin film (4), respectively, was reported. This concept was further generalized by tailoring integrated, electrically contacted, cofactor-dependent enzyme electrodes by the cross-linking of affinity complexes between NAD⁺-dependent enzymes and an electrocatalyst-NAD⁺ monolayer or thin film associated with electrodes.

Efficient electron transfer between redox-enzymes and conductive electrode supports as a result of structural alignment and optimal positioning of the electron mediators allowed development of non-compartmentalized biofuel cells (5). Cross-reactions of the anolyte fuel and catholyte oxidizer with the opposite electrodes were prevented due to the high specificity of the bioelectrocatalytic reactions at the electrodes, and thus the use of a membrane separating the catholyte and anolyte solutions could be eliminated. This kind of biofuel cells was suggested as a self-powered biosensor for glucose or lactate, since the output voltage and current signals are dependent on the substrate concentration (6).

Recently, efforts have been directed towards the development of functional metal or semiconductor nanoparticle-polymer hybrid systems exhibiting tailored sensoric, electronic, and photoelectrochemical functions. An example of a hybrid system is a copper-polyacrylic acid polymer that can be reversibly switched between electro-conductive and non-conductive states (7).

### SUMMARY OF THE INVENTION

Generally, the present invention relates to tunable and switchable electrode.

Thus, according to a first aspect, the present invention provides an electrode carrying on at least a portion of its support surface a hybrid polymer matrix (hereinafter abbreviated “HPM”), a catalyst that can catalyze a redox reaction and an optional electron mediator group that enhances the electrical contact between the HPM and the catalyst, the HPM being capable to be electrochemically changed from a non-conductive state to a conductive state. The HPM in its conductive state enables electrical contact between the electrode's elements and its support.

The electrode of the invention may be used in electronic devices, preferably as biocatalytic electrode. Examples of such uses are in fuel cells that preferably operate using fuels from biological systems and/or biological catalysts. Preferably, the fuel cell is a biofuel cell that operates using biological catalysts such as enzymes. It is to be noted that the terms fuel cell and biofuel cell are used interchangeably in the present application.

Generally, fuel cells operate with two electrodes, one being an anode and another one being a cathode. Nevertheless, according to the present invention, it is sufficient that only one of the two electrodes is of the switchable and tunable kind described above, whereas the second electrode is of a regular type.

However, in a preferred embodiment, the fuel cell of the invention is made of a pair of such tunable and switchable electrodes, one of the electrodes being an anode and the other a cathode. The anode carries on its surface a hybrid polymeric matrix (HPM) and a catalyst, e.g. an enzyme, capable of catalyzing an oxidation reaction. The HPM is capable to be electrochemically changed from a non-conductive state to a conductive state. In the non-conductive state the HPM preferably consists of negatively charged polymer matrix that electrostatically accommodates metal cations in the matrix. The HPM and the catalyst layers are bound either directly to each other or indirectly through an electron mediator group which can enhance the transfer of electrons between the HPM and the catalyst. Alternatively, the biocatalyst can be reconstituted on cofactor units bound to the HPM.

The cathode also carries on its surface an HPM that is identical to that on the anode and a catalyst capable of catalyzing the reduction of an oxidizer, preferably oxygen, to water. The catalyst is preferably an enzyme or enzyme-assembly. In addition, the cathode may also carry a mediator that enhances the electrical contact between the HPM and the catalyst. Alternatively, the cathode may carry cofactor units for the enzyme reconstitution providing the enzyme electrical contacting.

The HPM imparts to the electrode and thus to the fuel cell of the present invention the advantages of being both switchable and tunable. These properties are especially useful in implantable devices such as pacemakers, insulin pumps or any other power-supplying units. The switchable properties may be explained as follows:

The HPM associated with the electrodes may be electrochemically reduced to the metal⁰ (i.e. zero state)-polymer conductive state, while the oxidation of the conductive state during the operation of the fuel cell yields the non-conductive metal cation-polymer state. In the conductive state of the HPM, the biocatalytic systems are electrically contacted with the electrodes, thus allowing the fuel cell operation. In the non-conductive state of the HPM, the biocatalytic systems lack electrical contact with the electrodes, thus resulting in high electron transfer resistances switching “OFF” the fuel cell performance. The cyclic electrochemical switching “ON”and “OFF” of the fuel cell of the invention is achieved by reversible application of reductive potential and oxidative potential on the electrodes. This switching process allows the reversible activation and deactivation of the fuel cell operation as a power source or as a self-powered sensor.

It is to be noted that for the electrical contacting of the enzyme with the electrode it is required that the metal formation within the HPM proceed in a three-dimensional manner, through the entire HPM matrix. This is surprisingly achieved in the fuel cell of the invention since upon application of external reductive potential, three-dimensional metal clusters are formed that exhibit the appropriate dimensions and roughness that electrically connect between the enzyme and the electrode.

Application of the reductive potential for shorter time-intervals (i.e. time intervals that are shorter than that required for full reduction of HPM) results in the partial reduction of the HPM to the conductive state, thus allowing tuning of the fuel cell output. The tunable conductivity of the fuel cell of the invention is surprising, and implies a porous, dendritic, three-dimensional array of metal clusters. The impedance measurements performed on the fuel cell allow to correlate the electron transfer resistance values at the electrodes with the voltage-current and power-resistance functions of the fuel cell.

According to another aspect thereof, the present invention provides a novel fuel cell. The fuel cell comprises a pair of electrodes, one of the electrodes being an anode and the other a cathode, wherein both electrodes carry on at least a portion of their support surface a hybrid polymer matrix (HPM), a catalyst layer and an optional electron mediator group that enhances the electrical contact between the HPM and the catalyst. The HPM is capable to be electrochemically changed from a non-conductive state to a conductive state such that in its conductive state the catalyst layer is electrically contacted with the electrode support, thus allowing the fuel cell operation.

Preferably, the catalyst layer carried on the anode or cathode surface comprises a redox enzyme. The redox enzyme is cofactor-dependent, examples of the cofactor being flavin adenine dinucleotide phosphate (FAD), pyrroloquinoline quinone (PQQ), nicotinamide adenine dinucleotide (NAD), nicotinamide adenine dinucleotide phosphate (NADP), hemes and iron-sulfur clusters.

Examples of the enzyme carried on the anode electrode are glucose oxidase (GOx), glucose dehydrogenase, lactate dehydrogenase (LDH), fructose dehydrogenase, cholin oxidase, amino acid oxidase and alcohol dehydrogenase. Examples of the enzyme carried on the cathode electrode is selected from lacase, billirubin oxidase, and a complex formed of cytochrome c/cytochrome oxydase (COx).

The HPM is characterized by comprising in the non-conductive state a polymer carrying negatively charged groups that electrostatically accommodate metal cations. Examples of negatively charged groups are carboxyl, sulphonate, and phosphate, while examples of polymers that are suitable for use are polyacrylic acid, polylysine, polystyrene sulfonate, nafion, etc. The metal cations are preferably cations of transition metals, for example Cu, Ag, Hg, Cr, Fe, Ni, Zn. Preferably, the metal is copper.

Electrodes support suitable for use in the fuel cell of the present invention are made of conducting or semi-conducting materials, for example gold, platinum, palladium, silver, carbon, copper, indium tin oxide (ITO), etc. For invasive analyses the electrodes must be constructed of bio-compatible non hazardous substances, and fabricated as thin needles to exclude pain upon invasive penetration.

The fuel cell of the invention is usually used without a membrane between the electrodes and this is one of its benefits, especially when used in invasive applications. Nevertheless, the biosensor may also operate, when necessary, with a membrane.

The fuel cell of the invention may be used as a power supply for electrical devices. A method of powering an electrical device comprises the steps of electrically connecting the fuel cell of the invention to the device, electrooxidizing the fuel (e.g. glucose, etc.) at the anode and electroreducing an electron accepting molecule (e.g. oxygen) at the cathode, to generate electrical power. The internal switching properties of the electrode of the invention enable instant activation and deactivation of the power source and this is a major benefit thereof, especially when the electrical device is implanted within a human's body.

The fuel cell of the invention may also be used as a sensor, more specifically a biosensor. There is thus provided in the present invention, a biosensor that is self-powered by fluids that contain at least one substance capable to undergo biocatalyzed oxidation or reduction. The biosensor of the invention may be used in vivo as an implanted invasive device or ex vivo as a non-invasive device in the determination of the concentration and/or the identity of analytes in fluids of environmental, industrial, or clinical origin, e.g. blood tests, biocatalytic reactors, wine fermentation processes, etc.

In particular, the invention provides according to another aspect, a system for the determination of an analyte in a liquid medium comprising a self-powered biosensor and a detector for measuring an electrical signal (voltage or current) generated by the biosensor while the analyte is being oxidized or reduced. The analyte is capable of undergoing a biocatalytic oxidation or reduction in the presence of an oxidizer or reducer, respectively.

The term “determination” should be understood as meaning the measurement of the concentration and/or the presence of a substance.

The analytes that may be detected by the sensor of the invention are those capable to undergo biocatalytic oxidation or reduction reactions. Preferably, the analyte is usually an organic substance and the invention will be described herein below with reference to oxidizable organic analytes. Examples of such analytes are sugar molecules, e.g. glucose, fructose, inannose, etc; hydroxy or carboxy compounds, e.g. lactate, ethanol, methanol, forinic acid; amino acids or any other organic materials that serve as substrates for redox-enzymes.

According to another aspect, the present invention provides a method for determining an analyte in a liquid medium, said analyte being capable to undergo a biocatalytic oxidation or reduction reaction in the presence of an oxidizer or a reducer, respectively, the method comprising:

(i) providing a system comprising the biosensor of the invention and a detector for measuring an electrical signal generated by said biosensor while the analyte is being oxidized or reduced; (ii) activating the biosensor of the system by applying reductive potential to shift the HRM on both electrodes of the biosensor from non-conductive into a conductive state; (iii) contacting the activated biosensor of the system with the liquid medium; (iv) measuring the electric signal generated between the cathode and the anode, the electric signal being indicative of the presence and/or the concentration of said analyte; (v) determining the analyte based on said signal.

When the liquid medium is, for example, a body fluid e.g. blood, lymph fluid or cerebro-spinal fluid, and the method is carried out in an invasive manner, the method comprises inserting the biosensor into the body and bringing it into contact with the body fluid and determining the analyte in the body fluid within the body.

### BRIEF DESCRIPTION OF THE DRAWINGS

In order to understand the invention and to see how it may be carried out in practice, preferred embodiments will now be described, by way of non-limiting example only, with reference to the accompanying drawings, in which:

FIG. 1 schematically illustrates the electrochemical generation of the polyacrylic acid film on an Au electrode and the assembly of the integrated Cu²⁺-polymer film electrode.

FIG. 2 schematically illustrates the stepwise preparation of the biocatalytic anode, by covalent binding of PQQ and N6-(2-aminoethyl)-flavin adenin dinucleotide (FAD) to the polymer-functionalized electrode followed by the reconstitution of apo-glucose oxidase.

FIG. 3 schematically illustrates the stepwise preparation of the biocatalytic cathode, by covalent attachment of iso-2-cytochrome c (Cyt c) to the polymer-functionalized electrode surface using N-succinimidyl-3-maleimidopropionate (3) as a heterobifunctional linker, followed by affinity binding of cytochrome oxidase (COx) and the crosslinking of the protein complex layer.

FIG. 4A illustrates a biofuel cell configuration before assembling together all its parts.

FIG. 4B illustrates a biofuel cell configuration in assembled form.

FIG. 4C schematically shows a scheme for electrical measurements.

FIG. 5 illustrate electrochemical processes in the Cu²⁺/Cu⁰-polyacrylic acid hybrid thin film: FIG. 5A: a cyclic voltammogram of the Cu²⁺/Cu⁰-polyacrylic acid hybrid film, at potential scan rate 10 mV·s-1. FIG. 5B: cathodic current decay upon the application of a potential step from 0.5 V to −0.5 V on the Cu²⁺-polymer-functionalized electrode. Arrows a-e show time-interval applied for the electrochemical reduction of Cu2⁺ ions in the polymeric matrix. FIG. 5C: anodic current decay upon the application of a potential step from −0.5 V to 0.5 V on the Cu⁰-polymer-functionalized electrode. The measurements were performed in the presence of 0.1 TRIS-buffer, pH=7.0, in the cell under Argon.

FIG. 6 show the reversible switching “ON” and “OFF” of: (A) The short-circuit current, I_{sc}. (B) The open-circuit voltage, V_{oc}, generated by the biofuel cell.

FIG. 7 show the reversible activation and deactivation of the biocatalytic cathode and anode (7A and 7B, respectively) by the electrochemical reduction of the Cu²⁺-polymer film and the oxidation of the Cu⁰-polymer film, respectively.

FIG. 8 show the open-circuit voltage (V_{oc}) at a variable concentration of glucose injected into the biofuel cell device: FIG. 8A: after the anode and cathode of the biofuel cell were activated by the application of the potential corresponding to −0.5 V for 1000 s. FIG. 8B: after the anode and cathode of the biofuel cell were deactivated by the application of the potential of 0.5 V for 5 s. FIG. 8C: Calibration plots of the glucose sensing when the biofuel cell is activated (a) and deactivated (b).

FIG. 9A illustrates a graph which shows the current-voltage behavior of the biofuel cell at different external load resistances;

FIG. 9B illustrates a graph which shows the electrical power extracted from the biofuel cell at different external load resistances.

FIGS. 10 shows Nyquist plots (Zᵢₘ vs. Zᵣₑ) corresponding to the impedance spectra of the biofuel cell measured between the cathode and anode (two-electrodes mode) in the presence of 80 mM glucose solution saturated with air. FIG. 10A: The biofuel cell is in the “OFF” state after the potential of 0.5 V was applied on the two biocatalytic electrodes for 5 s. FIG. 10B: the biofuel cell is in the “ON” state after the potential of −0.5 V was applied on the both biocatalytic electrodes for 1000 s.

FIG. 11 shows Nyquist plots (Zᵢₘ vs. Zᵣₑ) corresponding to the impedance spectra of the biofuel cell measured between the cathode and anode (two-electrodes mode) in the presence of 80 mM glucose solution saturated with air after the reductive potential of −0.5 V was applied on the two biocatalytic electrodes for different time-intervals: (a) 200 s, (b) 400 s, (c) 600 s, (d) 800 s, and (e) 1000 s.

FIG. 12 shows Nyquist plots (Zᵢₘ vs. Zᵣₑ) corresponding the impedance spectra of: (a) the GOx-functionalized anode (three-electrodes mode), (b) the Cyt c/COx-functionalized cathode (three-electrodes mode), (c) the whole biofuel cell (two-electrodes mode). The measurements were performed in the presence of 80 mM glucose solution saturated with air, and after the biocatalytic electrodes were activated by the application of the potential of −0.5 V for 1000 s.

FIG. 13 illustrates a graph showing time-dependent open-circuit voltage, V_{oc}, generated by the biofuel cell in the presence of 80 mM glucose solution saturated with air.

### DETAILED DESCRIPTION OF THE INVENTION

The following specific embodiments are intended to illustrate the invention and shall not be construed as limiting its scope.

An electroswitchable and tunable biofuel cell based on the biocatalyzed oxidation of glucose is described. The anode is designed so as to consist of HPM, an electron-mediating layer and a catalyst layer. More specifically, the anode consists of Cu²⁺-polyacrylic acid film as the HPM, on which the redox-relay pyrroloquinoline quinone (PQQ) and the flavin adenine dinucleotide (FAD) cofactor are covalently linked. Apo-glucose oxidase is reconstituted on the FAD sites to yield the glucose oxidase (GOx)-functionalized electrode. The cathode consists of a Cu²⁺-polyacrylic acid film as the HPM, that provides the functional interface for the covalent linkage of cytochrome c (Cyt c) that is further linked to cytochrome oxidase (COx).

Electrochemical reduction of the Cu²⁺-polyacrylic acid films (applied potential −0.5 V vs. SCE) associated with the anode and cathode yield the conductive Cu⁰-polyacrylic acid matrices that electrically contact the GOx-electrode and the COx/Cyt c-electrode, respectively. The short-circuit current and open-circuit voltage of the biofuel cell correspond to 105 μA (current density ca. 550 μA·cm⁻²) and 120 mV, respectively, and the maximum extracted power from the cell is 4.3 μW at an external loading resistance of 1 kΩ.

The electrochemical oxidation of the polymer films associated with the electrodes (applied potential 0.5 V) yields the non-conductive Cu²⁺-polyacrylic acid films that completely block the biofuel cell operation. By the cyclic electrochemical reduction and oxidation of the polymer films associated with the anode and cathode between the Cu⁰-polyacrylic acid and Cu²⁺-polyacrylic acid states the biofuel cell performance is reversibly switched between “ON” and “OFF” states, respectively. In other words, the output power (voltage and current) can be reversibly switched between “ON” and “OFF” states and the magnitude of the voltage-current output can be precisely tuned by an electrochemical input signal.

The electrochemical reduction of the Cu²⁺-polymer film to the Cu⁰-polymer film is a relatively slow process (ca. 10-20 minutes) since the formation and aggregation of the Cu⁰-clusters requires the migration of Cu²⁺ ions in the polymer film and their reduction at conductive sites. The slow reduction of the Cu²⁺-polymer films allows controlling the content of conductive domains in the films and tuning the output power of the biofuel cell.

The electron transfer resistances of the cathodic and anodic processes may be characterized by impedance spectroscopy. Also, the overall resistances of the biofuel cell generated by the time-dependent electrochemical reduction process may be followed by impedance spectroscopy and correlated with the internal resistances of the cell upon its operation.

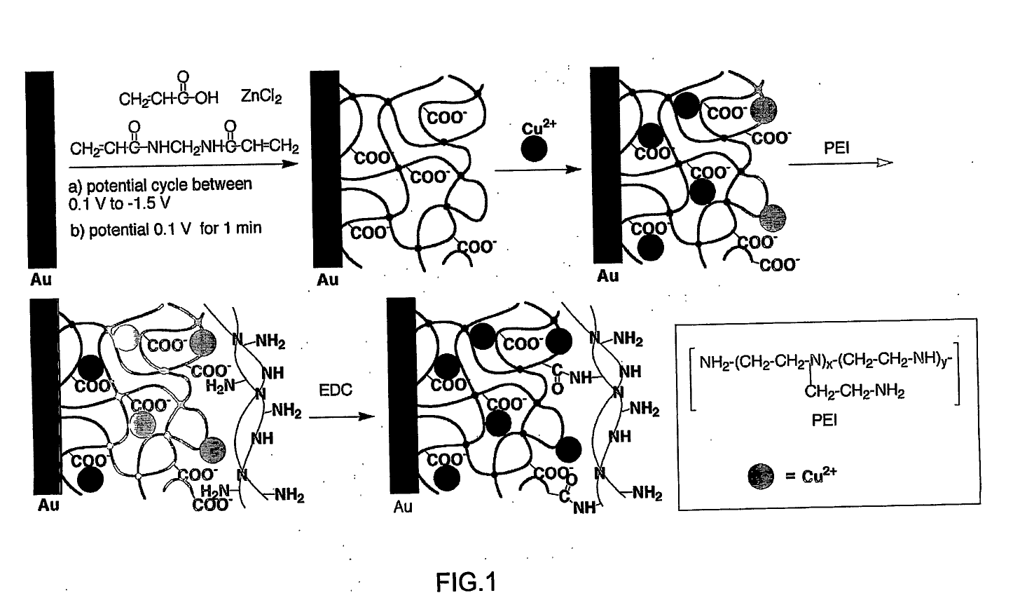

In a specific example, schematically showed in FIG. 1, a polyacrylic acid thin film was prepared by electropolymerization starting from acrylic acid as a monomer and methylene-bis-acrylamide as a cross-linker at a molar ratio of 50:1 were electropolymerized on gold electrodes (Au-covered glass slides) in the presence of ZnCl₂, 0.2 M, as catalyst. The electropolymerization was performed by potential cycling (5 cycles, 50 mV·s⁻¹) between 0.1 V and −1.5 V followed by application of 0.1 V for 1 minute. The co-deposited metallic zinc produced at the negative potentials was electrochemically dissolved at the potential of 0.1 V. The residual traces of Zn⁰ were dissolved in HCl and the produced Zn²⁺ cations were washed off. The polymeric film was characterized by surface plasmon resonance and the film thickness corresponds to ca. 280 nm (7).

The polymeric thin film was reacted with 0.1 M CuSO₄ solution for 1 hour to saturate the polymeric matrix with Cu²⁺ ions. Then the electrode surface was reacted with polyethyleneimine in the presence of a carbodiimide coupling reagent (EDC). This resulted, as schematically showed in FIG. 1, in the covalent attachment of the amine groups of polyethyleneimine (PEI) to the carboxylic groups of the polyacrylic acid film, thus yielding a positively charged capping layer preserving Cu²⁺ ions inside the polymeric matrix and providing amine functional groups for further modification of the electrode. The capping layer formed of polyethyleneimine is positively charged as a result of the amino groups of PEI that are protonated in an aqueous solution yielding positively charged ammonium groups. Microgravimetric quartz-crystal microbalance (QCM) measurements that follow the similar modification steps were performed on a QCM-electrode. These measurements reveal that the electrode surface loading with the polyacrylic acid film, the Cu²⁺ ions, and the polyethyleneimine layer correspond to 3.1×10⁻⁵ g·cm⁻², 4.5×10⁻⁶ g·cm⁻², and 1.2×10⁻⁶ g·cm⁻², respectively.

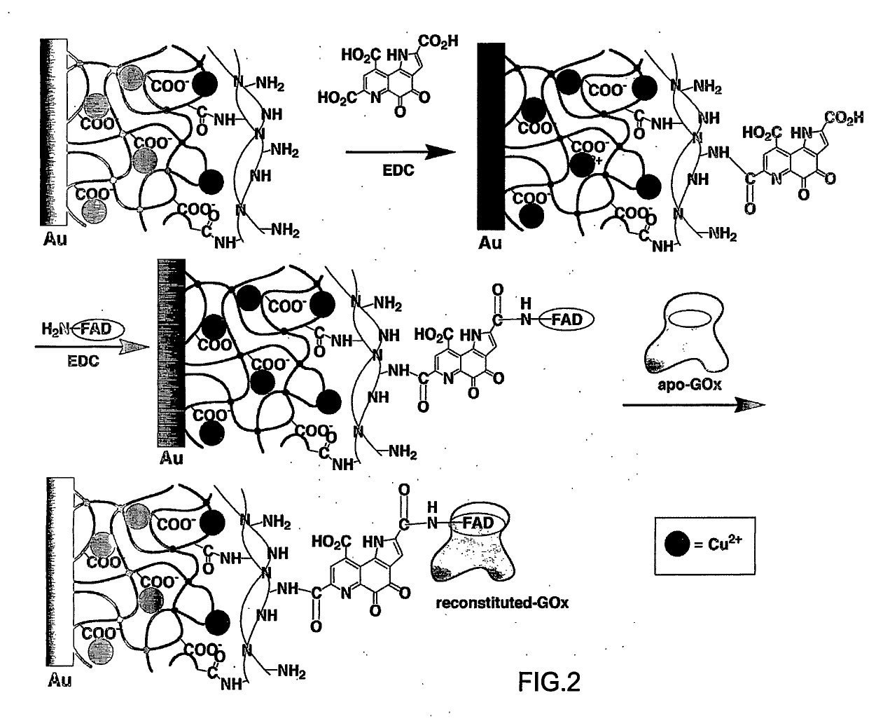

The polyacrylic acid Cu²⁺/polyethyleneimine-functionalized electrode was reacted with pyrroloquinoline quinone, (PQQ), and then with N⁶-(2-aminoethyl)-FAD, as schematically showed in FIG. 2. The PQQ-FAD dyad was then used to reconstitute apo-GOx with the FAD-cofactor and to provide mediated electron transfer via the PQQ-unit, thus yielding biocatalytic interface for the glucose oxidation. Quartz-crystal microbalance measurements for similar modification steps were performed on a QCM-electrode and reveal that the electrode loadings with PQQ, FAD and GOx correspond to ca. 2×10⁻¹⁰, 2×10⁻¹⁰, and 3×10⁻¹² mole·cm⁻², respectively. These values are similar to the random densely packed monolayer coverages.

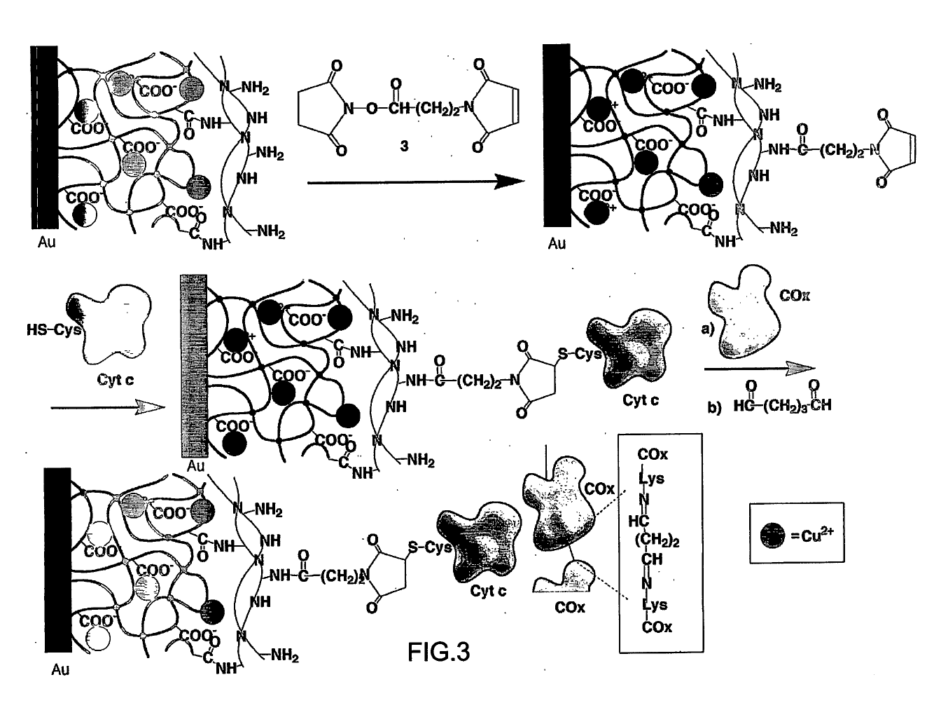

The preparation of the cathode used in the fuel cell of the invention is schematically showed in FIG. 3. Heterobifumctional reagent N-succinimidyl-3-maleimidopropionate 3 was applied to attach covalently the iso-2-cytochrome c (Cyt c) to the polymer film. The single cysteine residue of the Cyt c was covalently linked to the maleimide functional group providing alignment of the redox protein on the surface. Interaction of the cyt c-functionalized surface with cytochrome oxidase (COx) resulted in a stable affinity complex between Cyt c and COx (association constant Kₐ=1.2×10⁷ M⁻¹).⁴⁵ Crosslinking of the affinity complex with glutaric dialdehyde resulted in the integrated biocatalyst capable of reduction of O₂ to water, thus, yielding a biocatalytic cathode. Quartz-crystal microbalance measurements for similar modification steps were performed on a QCM-electrode, and these reveal that the electrode loadings with Cyt c and COx are ca. 1×10⁻¹¹ and 3×10⁻¹² mole·cm⁻², respectively. These surface densities correspond to a random densely packed Cyt c and COx monolayer configuration.

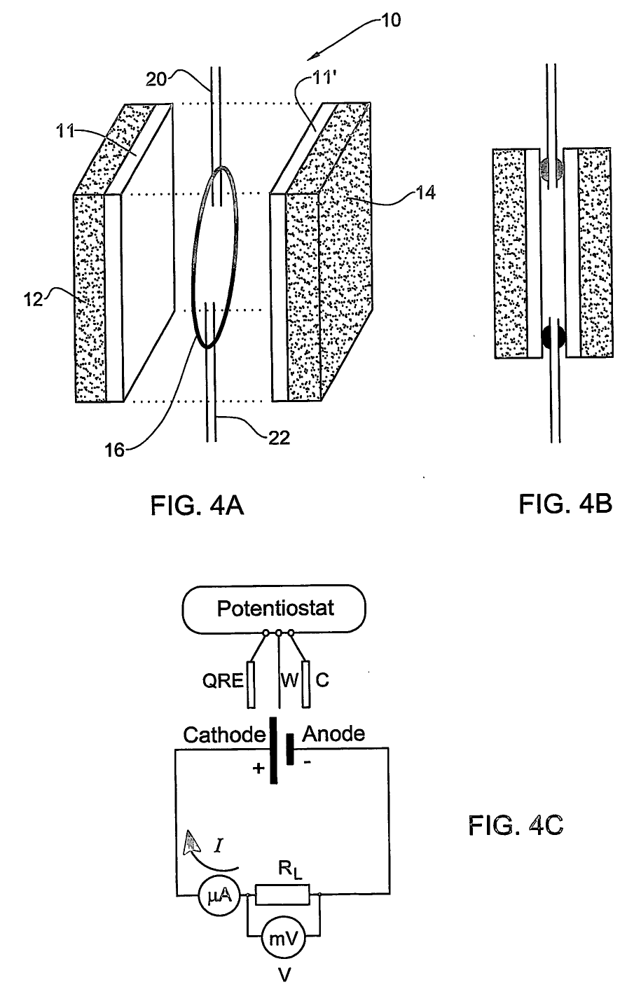

The Cyt c/COx-functionalized electrode and the PQQ-FAD/GOx-functionalized electrode were assembled as a cathode and anode, respectively, in a fuel cell configuration. Reference is being made to FIG. 4A that schematically show a simple configuration of a biosensor that may be used in the system of the invention. However, many other assemblies may be fabricated, that are based on the concept of the present invention. Thus, FIG. 4A shows a fuel cell 10 (before assembling together all its parts) organized as a flow-injection cell that consists two enzyme-functionalized Au-electrodes (ca. 0.19 cm active area), acting as anode 11 and cathode 11′. Both electrodes are supported on glass plates 12 and 14 and are separated by a rubber 0-ring 16 (ca. 2 mm thickness). Inlet needle 20 and outlet needle 22 implanted into the rubber ring convert the unit into a flow cell, where a liquid medium may flow at a flow rate of 1 min The distance between the cathode and the anode is ca. 2 mm. FIG. 4B shows the same device in assembled form. The electrical measurements were carried out by the scheme illustrated in FIG. 4C. According to this scheme, the biofuel cell output voltage and current are measured on the external variable load resistance R_{L}, using an electrometer. The electrochemical measurements were performed on the cathode or anode of the cell connected to the working electrode inlet of the potentiostat W. Two metallic needles are used as a counter electrode, C and a quasi-reference electrode QRE.

It should be noted that the device shown in FIG. 4A operates without a membrane and this is a significant advantage, especially for invasive applications, since this possibility renders the device configuration much simpler.

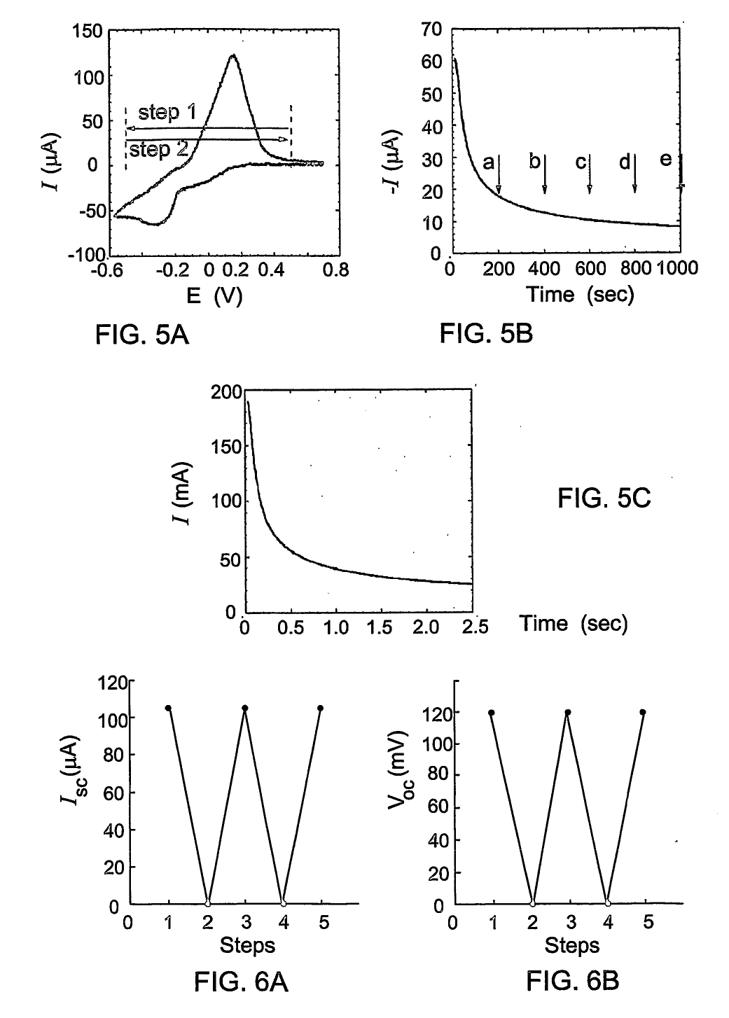

FIG. 5(A) shows the cyclic voltammogram of the polyacrylic acid/Cu²⁺/polyethyleneimine-functionalized electrode modified with the biocatalytic system (Cyt c/COx or PQQ-FAD/GOx) when the cell was loaded with a background electrolyte only (0.1 M TRIS-buffer, pH=7.0, deaerated with Ar). The cyclic voltammogram was recorded using two metallic needles implanted into the cell as a counter electrode and a quasi-reference electrode. This cyclic voltammogram follows the known mechanism of the copper redox process (8). Upon sweeping the potential from 0.7 k V to −0.6 V a poorly resolved cathodic wave corresponding to the reduction of Cu²⁺ ions to Cu⁺ ions is observed at E_{pc1}=−0.05 V followed by the reduction wave of Cu⁺ to Cu⁰ at E_{pc2}=−0.3 V. Upon sweeping the potential back from −0.6 V to 0.7 V the reverse anodic peak is observed at Eₚₐₗ=0.18 V, corresponding to the oxidation of Cu⁰ to Cu²⁺. The intermediate redox state Cu⁺ is not observed because it undergoes disproportionation. Coulometric analysis of the redox waves recorded with a relatively fast potential scan rate (10 mV·s⁻¹) yields the amount of Cu²⁺/Cu⁰ that participates in the redox process upon the potential scan (ca. 40 s). The amount of redox active copper found from the cyclic voltammogram is ca. 400 ng·cm⁻², which is almost an order of magnitude smaller than the total amount of copper derived from the microgravimetric measurements. This discrepancy originates from slow charge propagation across the polymeric matrix, therefore on the time-scale of the cyclic voltammetry only the Cu²⁺ ions adjacent to the conductive support participate in the redox process.

The kinetics of the electrochemical reduction of Cu²⁺ ions across the polymeric matrix and the backward electrochemical oxidation of Cu⁰ metallic particles, were performed by chronoamperometric measurements and are showed in FIG. 5B, which shows the cathodic current decay upon the potential step from 0.5 V to −0.5 V. The kinetics of the reductive process, τ_{1/2}≈50 s, corresponds to the formation of the conductive aggregates of Cu⁰ particles across the polymeric matrix. Without being bound to theory it is supposed that the the slow kinetics of this process is attributed to the fact that the Cu²⁺ ions have to migrate through the polymer film and reach the electrode surface in order to be reduced. Upon this reductive process the conductive aggregates of Cu⁰ nanoparticles are growing from the electrode surface and penetrating the polymer film. The amount of the reduced Cu⁰ was derived by the integration of the cathodic current and corresponded to 4.4 μg·cm⁻² (6.9×10⁻⁸ mole·cm⁻²) after 1000 s of the reductive process. This surface loading is similar to that found by the quartz-crystal microbalance measurements. Taking into account the polymer film thickness of ca. 280 nm, as derived from the SPR measurements, the concentration of the redox active Cu²⁺/Cu⁰ in the film was calculated to be ca. 0.16 g·cm⁻³(2.5×10⁻³ mole·cm⁻³). The reductive process could be stopped at different time-intervals (shown with arrows a-e in FIG. 5B, providing various extents of the Cu²⁺ reduction and thus yielding different conductivities of the Cu⁰-polymeric matrix.

FIG. 5C shows the fast anodic current decay, τ_{1/2}≈0.2 s, upon the potential step from −0.5 V to 0.5 V after the potential of −0.5 V was applied to the electrode for 1000 s. Without being bound to theory it is supposed that the fast kinetics of this oxidative process (the oxidation of Cu⁰ to Cu²⁺) originates from the fact that the conductive assembly of the aggregated Cu⁰ particles is already produced across the polymeric matrix prior to the potential step, thus providing the electrochemical contact of all the Cu⁰ species. The amount of the oxidized copper generated in the anodic process is derived by the integration of the anodic current and it is similar to the amount of the reduced copper formed in the reductive process (ca. 4.4 μg·cm⁻²).

While the Cu²⁺-polyacrylic acid revealed very high resistance (transverse resistance between an Au 0.5 mm-diameter conductive tip and the electrode support, ca. 300 kΩ), the Cu⁰-polyacrylic acid film exhibited lower resistance (ca. 2.2 kΩ). These properties of the Cu²⁺/Cu⁰-polyacrylic acid film suggest that the electrical contact between the electrode support and the redox biocatalyst associated with the film could be electrically switched and tuned by controlling the resistance of the polymer medium. In order to study the effect of the redox state of the Cu²⁺/Cu⁰-polyacrylic acid film on the fuel cell output, the biocatalytic cathode and anode were preconditioned at the potentials of −0.5 V for 1000 s or at 0.5 V for 5 s to generate the reduced Cu⁰ or oxidized Cu²⁺ in the film, respectively. The voltage and current (V_{oc} and I_{sc}) produced by the fuel cell in these two states were measured in the presence of 80 mM glucose solution saturated with air.

FIG. 6 shows the reversible activation and deactivation of the fuel cell upon the formation of Cu⁰ state and Cu²⁺ state, respectively. The cell output is switched “ON” (steps 1, 3 and 5) by the application of the potential of −0.5 V to the both biocatalytic electrodes for 1000 s and switched “OFF” (steps 2 and 4) by the application of a potential of 0.5 V to the two biocatalytic electrodes for 5 s. The measurements were performed in the presence of 80 mM glucose solution saturated with air.

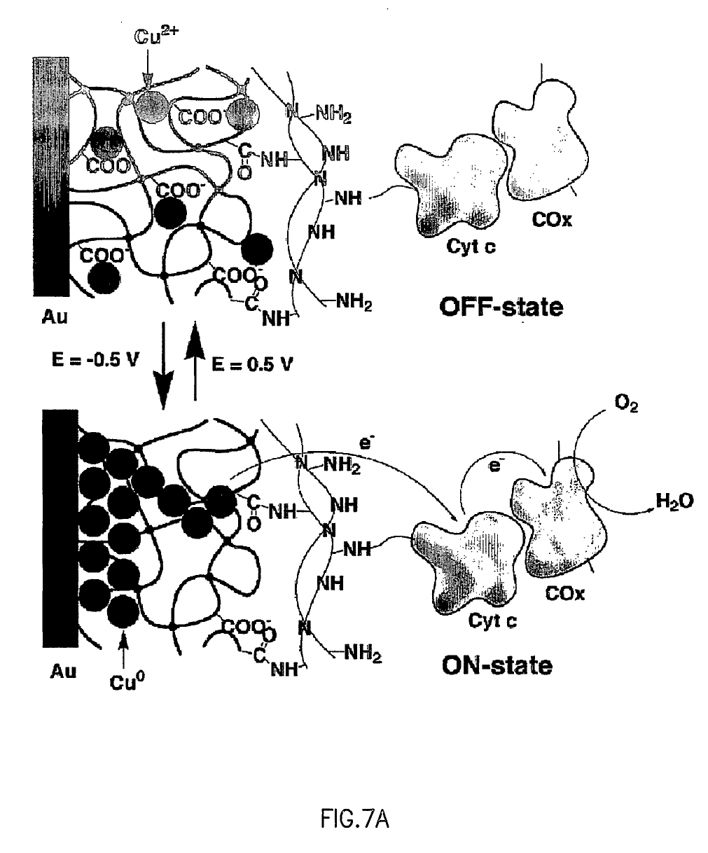

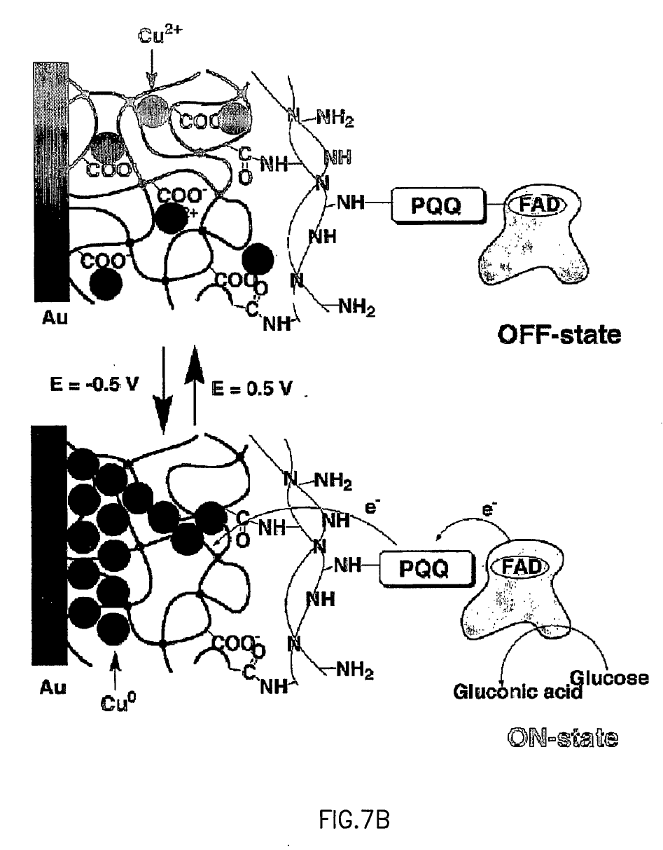

The fuel cell short-circuit current, as showed in FIG. 6A is ca. 105 μA (current density ca. 550 μA·cm⁻²) in the active state (Cu⁰-polyacrylic acid) and 0 μA in the non-active state (Cu²⁺-polyacrylic acid). The open-circuit voltage produced by the active state of the cell, as showed in FIG. 6B is ca. 120 mV and 0 mV in the Cu²⁺-polyacrylic acid deactivated state of the cell. It is believed that this effect is attributed to the fact that in the reduced state, the Cu⁰ nanoparticles generate the conductive aggregates penetrating through the polymeric matrix and providing electrical contacting of the biocatalyst with the electrode support. When the ionic state Cu²⁺ is electrochemically produced in the polymeric matrix, the biocatalysts are electrically disconnected from the electrode support and the biocatalytic process cannot yield the voltage and current formation across the cell. Thus, the complete switching “ON” and “OFF” was achieved for the biofuel cell upon conditioning the biocatalytic electrodes at the reductive potential of −0.5 V for 1000 s and at the oxidative potential of 0.5 V for 5 s, respectively. FIG. 7A schematically shows the reversible activation and deactivation of the biocatalytic cathode by electrochemical reduction of the Cu²⁺-polymer film and the oxidation of the Co⁰-polymer film, while FIG. 7B shows the similar activation and deactivation processes carried on the anode. It should be noted that both electrodes (the cathode and anode) are activated by the application of the reductive potential of −0.5 V in order to activate the entire biofuel cell, while application of the oxidative potential of 0.5 V on any of the biocatalytic electrodes results in the biofuel cell deactivation.

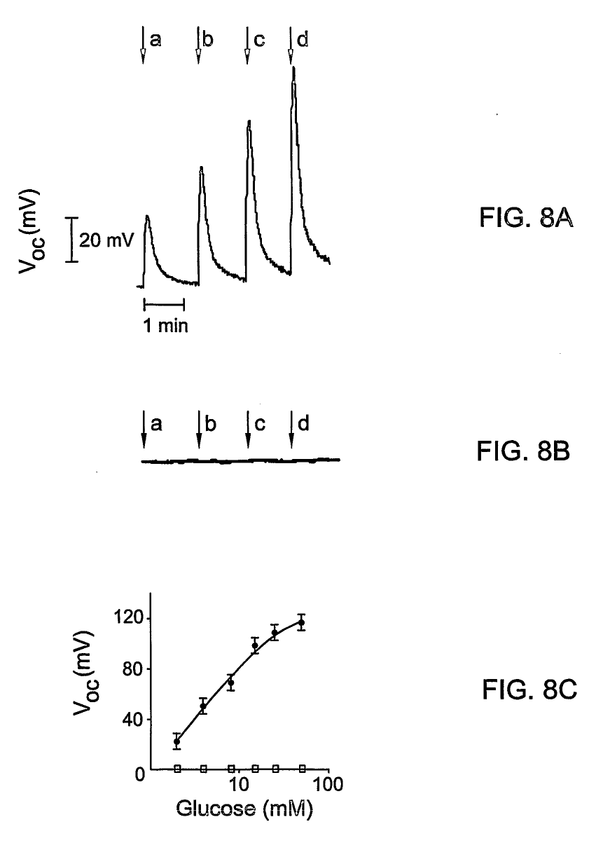

FIG. 8A illustrates the relation between the output voltage of the cell and the fuel concentrations. Accordingly, the output voltage signal is controlled by the glucose concentrations in the system, when the biocatalytic electrodes are activated to the conductive state by their preconditioning at the potential of −0.5 V for 1000 s. Injections of air-saturated solutions with the different glucose concentrations resulted in the variable voltage signals generated by the cell, thus allowing the glucose sensing. Arrows show the injections of glucose with the concentrations of: (a) 2 mM, (b) 3 mM, (c) 8 mM, (d) 40 mM.

The voltage output increases as the concentration of glucose is elevated. However, when any of the biocatalytic electrodes (the anode or cathode) is deactivated by the application of the oxidative potential of 0.5 V for 5 s, the cell voltage output is blocked to any glucose concentration and thus, the glucose biosensor is switched “OFF” as showed in FIG. 8B. The calibration plots for the self-powered glucose biosensor when it is in the “ON” state, curve (a), and in the “OFF ” state, curve (b) are showed in FIG. 8C. In all measurements the glucose solution was equilibrated with air.

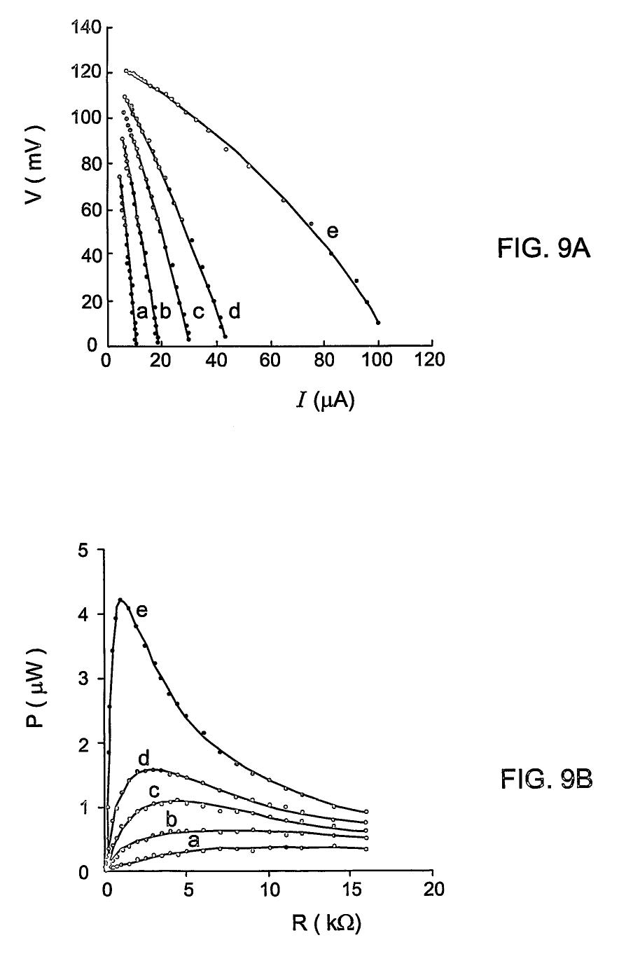

The slow kinetics characteristic to the reduction of the matrix and its transformation to the conductive medium allow us to terminate the process at different time-intervals and to achieve variable degrees of conductivity of the film. The controlled conductivity of the film could then be used to tune the voltage-current output of the biofuel cell. The reductive process was terminated after 200 s, 400 s, 600 s, 800 s, and 1000 s resulting in different voltage-current outputs of the cell. FIG. 9A shows the voltage-current curves of the biofuel cell in the presence of 80 mM glucose solution saturated with air. The voltage-current curves were measured at variable loading resistances (loading function) after the application of the reduction process on the electrodes for different time-intervals. It can be seen that the voltage-current output of the biofuel cell becomes higher when the reductive process applied on the Cu²⁺/polyacrylic acid film is longer. The reductive process performed for 1000 s resulted in the highest output values.

FIG. 9B shows the electrical power produced by the biofuel cell at variable resistances after application of the reductive potential on the biocatalytic electrodes for the different time-intervals. Curves a-e show the biofuel cell output functions after the reductive potential of −0.5 V was applied to the biocatalytic electrodes for different time-intervals: (a) 200 s, (b) 400 s, (c) 600 s, (d) 800 s, and (e) 1000 s. The measurements were performed in the presence of 80 mM glucose solution saturated with air.

It can be seen that the power output from the biofuel cell is smaller as the time-interval for the reduction of the Cu²⁺-polymer film to the Cu⁰-polymer film is shorter. Also, it was observed that the output power is less dependent on the value of the external resistances as the time-interval for the generation of the Cu⁰-polymer film is shorter. As the maximum value of the power output should occur at the external resistance load that is equal to the internal cell resistance, the results imply that at shorter time-intervals for the generation of the Cu⁰-polymer film the cell resistance is higher. Without being bound to theory, this conclusion may be explained by the fact that at shorter time-intervals for generating the Cu⁰ -polymer a substantial amount of the polymer film exists in a non-conductive state with high resistance and the biocatalysts in these polymer domains are inactive. This conclusion finds further support in impedance measurements.

When the reductive process that yields the Cu⁰state is longer, the conductivity of the hybrid film is increased and the electrical contacting of the biocatalysts and the electrodes is improved. This results in the decrease of the electron transfer resistance of the biocatalytic electrodes and yields smaller internal resistance of the biofuel cell. It should be noted that the internal resistance of the biofuel cell represents mainly the electron transfer resistance of the biocatalytic electrodes. As the time-interval for the reduction of the Cu²⁺-polymer film is shorter the content of electrically contacted biocatalyst with the electrode is lower and thus the average electron transfer resistance is higher. The smaller internal resistance of the cell allows the higher voltage and current outputs, but results in the sharp dependence of the produced power on the loading resistance values. Thus, variation of the reductive time-intervals applied to the biocatalytic electrodes allows the tuning of the output functions of the biofuel cell due to the change of the internal resistance of the cell.

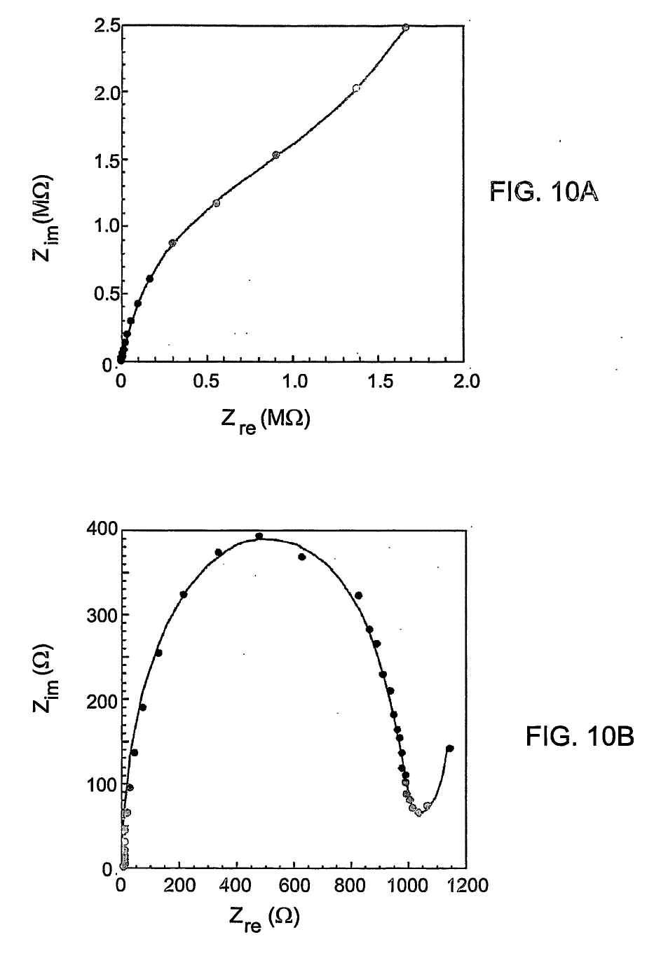

The mechanism suggested for the electrochemical switching of the biofuel cell between “ON” and “OFF” states was further supported by Faradaic impedance measurements. FIG. 10 shows the impedance spectra measured between the biocatalytic electrodes (two-electrodes mode) in the presence of 80 mM glucose solution saturated with air. FIG. 10A shows the impedance spectrum of the cell after the biocatalytic electrodes were deactivated by the application of the oxidative potential of 0.5 V for 5 s. The low frequency (0.1 Hz-1 Hz) impedance domain shows very high impedance values (Zᵢₘ and Zᵣₑ) of ca. 1-2 MΩ. Under this condition the biofuel cell does not generate any measurable voltage-current output. FIG. 10B shows the impedance spectrum of the cell after the biocatalytic electrodes were fully activated by the application of the reductive potential of −0.5 V for 1000 s. The diameter of the semi-circle domain of the spectrum corresponds to the overall electron transfer resistance of the biofuel cell, Rₑₜ≈1 kΩ. This value is similar to the value of the external loading resistance that provides the maximum power produced by the fully activated biofuel cell, as showed in FIG. 9B, curve (e). It should be noted that the maximum power output is achieved at the external loading resistance equal to the internal resistance of the battery (or fuel cell). Thus, the electron transfer resistance, Rₑₜ, derived from the impedance spectrum, as showed in FIG. 10B, corresponds to the internal resistance of the biofuel cell that operates in the fully activated state of the Cu⁰-polyacrylic acid-functionalized electrodes.

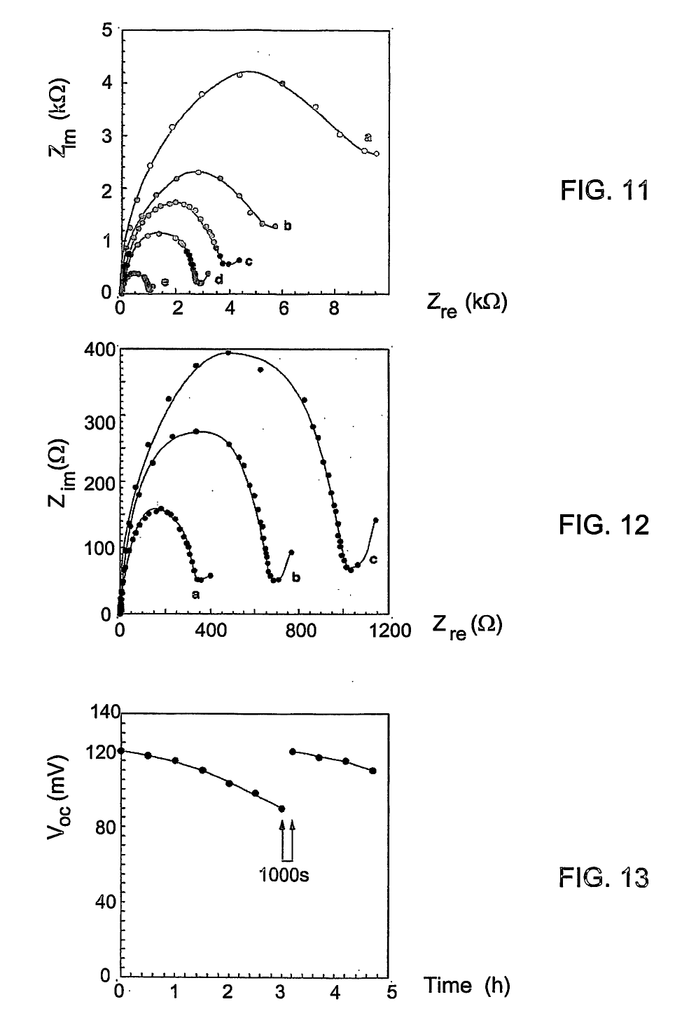

FIG. 11 shows the Faradaic impedance spectra measured between the biocatalytic electrodes (two-electrodes mode) upon operation of the biofuel cell after the reductive potential of −0.5 V was applied to the electrodes for different time-intervals. Curve (e) shows the impedance spectrum corresponding to the fully activated biofuel cell after application of the reductive potential of −0.5 V for 1000 s. Curves (a-d) show the impedance spectra corresponding to the partially activated biofuel cell after the reductive potential of −0.5 V was applied on the electrodes for 200 s, 400 s, 600 s, and 800 s, respectively. These spectra, at curves (a-d), correspond to the intermediate tunable states of the biofuel cell that operates between the fully deactivated state, showed in FIG. 10A, and the fully activated state, showed in FIG. 10B. The electron transfer resistances derived from the spectra showed in FIG. 11, curves (a)-(d), correspond to ca. 12 kΩ, 6 kΩ, 4 kΩ, 2.7 kΩ, respectively. Thus, the electron transfer resistances of the biofuel cell in its different degrees of electrochemical activation represent the internal resistances of the respective activated cells under operating conditions.

The overall electron transfer resistance of the fuel cell derived from the impedance spectrum measured between the cathode and anode (two-electrodes mode) is composed of the partial electron transfer resistances of the cathode and the anode that were measured separately (three-electrodes mode). The later measurements were performed for each of the biocatalytic electrodes using a counter electrode and a quasi-reference electrode in the cell, and is schematically showed in FIG. 12. Curve (a), (three-electrodes mode) shows the impedance spectrum of the GOx-functionalized anode in the presence of 80 mM glucose solution saturated with air after the electrode was preconditioned at the potential of −0.5 V for 1000 s. The electron transfer resistance of 340 Ω is derived from this spectrum. Curve (b), (three-electrodes mode) shows the impedance spectrum of the Cyt c/COx-functionalized cathode in the presence of 80 mM glucose solution saturated with air after the electrode was preconditioned at the potential of −0.5 V for 1000 s. The electron transfer resistance of 660Ω is derived from this spectrum. The overall electron transfer resistance of the biofuel cell measured between the anode and cathode (two-electrodes mode) is ca. 1000Ω, and this value fits nicely the sum of the electron transfer resistances of the cathode and anode measured separately, as predicted theoretically.

From the above impedance measurements one may conclude that the main contribution to the biofuel cell electron transfer resistance originates from the electron transfer resistance of the Cyt c/COx-functionalized cathode. Thus, the cathodic biocatalytic process represents the limiting step in the whole biofuel cell operation.

The biofuel cell operational stability has also been tested. Since a positive potential is generated on the biocatalytic cathode upon the cell operation, the conductive Cu⁰-state could be degraded due to the copper oxidation, thus resulting in the biofuel cell gradual deactivation. FIG. 13 shows the biofuel cell voltage output (V_{oc}) measured upon continuous cell operation in the presence of 80 mM glucose solution saturated with air pumped through the cell with the flow rate of 1 mL·min⁻¹. The open-circuit voltage slowly decreases from 120 mV to 90 mV after 3 hours of continuous operation. Arrows show the time-interval when the cell was re-activated by the application of the potential of −0.5 V on the biocatalytic cathode for 1000 s. After that the reductive potential of −0.5 V was applied for 1000 s to the biocatalytic cathode resulting in full re-activation of the cell and restoring the original V_{oc}=120 mV. From this result it may be assumed that the gradual decrease of the cell output originates from the partial oxidation of the conductive Cu⁰-polymeric matrix associated with the cathode, rather than from the degradation of the enzyme-biocatalytic systems. The biofuel cell performance could be maintained with no efficiency loss for at least 48 hours by the sequential re-activation steps that involve the application of the reductive potential on the cathode every 3 hours.

It should be emphasized that the switchable and tunable operation described above in connection with biofuel cells, applies to fuel cells in general.

In addition, when dealing with a biofuel cell, the biofuel cell may be composed of different biocatalysts, where glucose oxidase and cytochrome oxidase are specific examples. Also, the polymer film with metal ions providing switchable and tunable properties could be composed of various polymeric materials, preferably polyelectrolytes, where polyacrylic acid mentioned above is a specific example thereof. Concerning the metal ions that are electrochemically reduced and oxidized within the polymeric film in order to provide the switchable and tunable properties, these may be of different transition metals, for example Cu, Fe, Co, Ag, Ni, etc., where Cu is only a specific example thereof.

### EXAMPLES

Chemicals. Glucose oxidase (GOx, EC 1.1.3.4 from Aspergillus niger) was purchased from Sigma and used without further purification. Apo-glucose oxidase (apo-GOx) was prepared by a modification of the reported method (9). Cytochrome oxidase (COx) was isolated from a Keilin-Hartree heart muscle and purified according to a published technique (10). Yeast iso-2-cytochrome c (Cyt c) from Saccharomyces cerevisiae (Sigma) was purified by ion-exchange chromatography. N⁶-(2-Aminoethyl)-flavin adenine dinucleotide was synthesized and purified. All other chemicals, including pyrroloquinoline quinone (PQQ), acrylic acid, methylene-bis-acrylamide, N-succinimidyl-3-maleimidopropionate, 4-(2-hydroxyethyl)piperazine-1 ethanesulfonic acid sodium salt APES), tris(hydroxymethyl)aminomethane hydrochloride (TRIS), 1-ethyl-3-(3-dimethylaminopropyl)carbodiimide (EDC), glutaric dialdehyde, β-D-(+)-glucose were purchased from Sigma and Aldrich and used as supplied. Ultrapure water from Seralpur Pro 90 CN source was used in all experiments.

Modification of electrodes. Glass supports (TF−1 glass, 20×20 mm) covered with a Cr thin sublayer (5 nm) and a polycrystalline Au layer (50 nm) supplied by Analytical-μSystem (Germany) were used as conductive supports. These electrodes were modified with a polyacrylic acid thin film using the electropolymerization technique (11). The electropolymerization was performed in the aqueous solution composed of acrylic acid sodium salt, 2 M, methylene-bis-acrylamide, 0.04 M, and ZnCl₂, 0.2 M, pH=7.0, upon application of 5 potential cycles (50 mV·s⁻¹) between 0.1 V and −1.5 V. Then the potential of 0.1 V was applied for 1 minute to dissolve electrochemically metallic zinc produced in the film upon the electrochemical polymerization. The polymer-modified electrode was reacted with 0.1 M HCl for 2 minutes to dissolve residual amounts of metallic zinc, and then the electrode was washed with water and ethanol to clean the modified surface from Zn²⁺ ions and the excess of monomers. The polymer-modified electrodes were soaked in 0.1 M CuSO₄solution for 1 h in order to saturate the polyacrylic film with Cu²⁺ ions, and then the electrode surface was briefly washed with water. The modified electrodes were further reacted with a solution of polyethylenimine (M.W. 60,000) (5% v/v) in 0.1 M HEPES-buffer, pH=7.2, in the presence of EDC, 1×10⁻² M, for 1 h, and then washed with water. The polymer-modified electrode was incubated for 2 h in a 3 mM solution of PQQ (1) in 0.1 M HEPES-buffer, pH=7.2, in the presence of 5×10⁻³ M EDC, yielding the PQQ-functionalized surface. The covalent coupling of the N⁶-(2-aminoethyl)-FAD, (2), to the PQQ-modified electrode was performed by soaking the electrode in the 0.1 M HEPES-buffer solution (pH=7.2) containing 5×10⁻⁴ M (2) and 5×10⁻³ M EDC for 2 h at room temperature. The PQQ-FAD-functionalized electrode was reacted with 1 mg·mL⁻¹ apo-GOx in 0.1 M phosphate buffer, pH=7.0, for 5 h at room temperature. The modified electrode was washed with water to yield the GOx-reconstituted electrodes for biocatalytic oxidation of glucose. Another polymer-modified electrode was reacted with a 1×10⁻³ M solution of N-succinimidyl-3-maleimidopropionate (3) in 0.1 M HEPES-buffer, pH=7.2, for 2 h, followed by rinsing with water. The maleimide-functionalized electrode was treated with Cyt c solution, 0.1 mM, in 0.1 M HEPES-buffer, pH 7.2, for 2 h, followed by rinsing with water. To produce the integrated Cyt c/COx bioelectrocatalytic electrode for O₂ reduction, the resulting Cyt c-modified electrode was interacted with cytochrome oxidase (COx), 0.5 mM, in TRIS-buffer, pH 8.0, for 2 h, washed briefly with water and then treated with aqueous solution of glutaric dialdehyde, 10% v/v, for 30 min. The resulting modified electrode was washed with water.

Biofuel cell and electrochemical measurements. FIG. 4A shows the biofuel cell configuration. The system consists of two enzyme-functionalized electrodes (ca. 0.19 cm² active area) separated by a rubber O-ring (ca. 2 mm thickness). The first electrode functionalized with the reconstituted GOx and the second electrode functionalized with Cyt c/COx assembly are acting as anode and cathode, respectively. Two metallic needles (inlet and outlet) implanted into the rubber ring convert the unit into a flow cell (flow rate 1 mL·min⁻¹). A peristaltic pump was applied to control the flow rate. Glucose solutions in 0.1 M TRIS-buffer, pH=7.0, saturated with air were applied to power the biofuel cell. The needles were also used as a counter electrode and a quasi-reference electrode when electrochemical measurements were performed for each of the biomaterial-functionalized electrodes in the cell. The quasi-reference electrode was calibrated according to the potential of dimethyl viologen, E^{o}=−0.687 V versus SCE, measured by cyclic voltammetry, and the potentials are reported versus SCE. Cyclic voltammetry and chronoamperometry experiments were performed using an electrochemical analyzer (EG&G model 283) linked to a computer (EG&G software 270/250). Impedance measurements were performed using an electrochemical analyzer composed of a potentiostat/galvanostat (EG&G, model 283) and frequency response detector (EG&G model 1025) connected to a computer (EG&G software PowerSuite 2.11.1). The impedance measurements were performed in the frequency range of 100 mHz to 50 kHz between the cathode and anode of the biofuel cell (two-electrodes mode) and for each biocatalytic electrode using a counter electrode and a quasi-reference electrode (three-electrodes mode). The experimental impedance spectra were simulated using electronic equivalent circuits. For this purpose commercial software (ZView version 2.1b, Scribner Associates, Inc.) was employed. Voltage and current produced by the biofuel cell were measured on a variable external resistance using an electrometer (Keithley 617), FIG. 4B.

Microgravimetric Quartz-Crystal Microbalance (QCM) Measurements. A QCM analyzer (Fluke 164T multifunction counter, 1.3 GHz, TCXO) and quartz crystals (AT-cut, 9 MHz, Seiko) sandwiched between two Au electrodes (area 0.2±0.01 cm², roughness factor ca. 3.5) were employed for the microgravimetric analyses of the modified electrodes in air. The QCM crystals were calibrated by electropolymerization of aniline in 0.1 M H₂SO₄ and 0.5 M Na₂SO₄ electrolyte solution, followed by coulometric assay of the resulting polyaniline film and relating of the crystal frequency changes to the electrochemically derived polymer mass.

1-44. (canceled)

45. An electrode carrying on at least a portion of its support surface a hybrid polymer matrix (HPM), a catalyst that can catalyze a redox reaction and an optional electron mediator group that enhances electrical contact between the HPM and the catalyst, said HPM being capable to be electrochemically charged from a non-conductive state to a conductive state.

46. The electrode according to claim 45, wherein said HPM in its conductive state enables electrical contact between said electrode and said catalyst.

47. The electrode according to claim 45 wherein said catalyst layer carried on the electrode surface comprises a redox enzyme.

48. The electrode according to claim 45, wherein said HPM comprises in the non-conductive state a polymer carrying negatively charged groups that electrostatically accommodate metal cations.

49. The electrode according to claim 48 wherein said negatively charged groups are selected from carboxyl, sulphonate and phosphate.

50. The electrode according to claim 48 wherein said polymer is selected from polyacrylic acid, polylysine, polystyrene sulfonate and nafion.

51. The electrode according to claim 48 wherein said metal cations are cations of transition metals.

52. The electrode according to claim 51 wherein said metal cations are cations of metals selected from Cu, Ag, Hg, Cr, Fe, Ni and Zn.

53. The electrode of claim 45 having switchable conductivity properties such that in the conductive state of the HPM, the catalyst is electrically contacted with the electrode' support, while in the non conductive state of the HPM, the catalyst lacks electrical contact with the electrode' support, thus resulting in high electron transfer resistances.

54. The electrode of claim 45, having tunable conductivity properties such that application of reductive potential for time-intervals that are shorter than that required for full reduction of HPM, results in the partial reduction of the HPM to the conductive state, thus allowing tuning of the electrode's output.

55. The electrode of claim 45, wherein said HPM is capable to be changed from a non-conductive to a conductive state and vice versa by reversible application of reductive potential and oxidative potential on the electrode.

56. A fuel cell comprising at least one electrode according to claim 45.

57. The fuel cell according to claim 56 comprising a pair of electrodes, one of the electrodes being an anode and the other a cathode, wherein both electrodes carry on at least a portion of their support surface a hybrid polymer matrix (HPM), a catalyst layer and an optional electron mediator group that enhances the electrical contact between the HPM and the catalyst, said HPM being capable to be electrochemically charged from a non-conductive state to a conductive state such that in its conductive state the catalyst layer is electrically contacted with the electrode that carries it, thus allowing the fuel cell operation.

58. The fuel cell of claim 57, wherein said catalyst layer carried on the anode or cathode surface comprises a redox enzyme.

59. The fuel cell of claim 58, wherein said redox enzyme is cofactor-dependent, the cofactor being selected from flavin adenine dinucleotide phosphate (FAD), pyrroloquinoline quinone (PQQ), nicotinamide adenine dinucleotide (NAD), nicotinamide adenine dinucleotide phosphate (NADP), hemes and iron-sulfur clusters.

60. The fuel cell of claim 58, wherein the enzyme carried on the anode electrode is selected from glucose oxidase (GOx), glucose dehydrogenase, lactate dehydrogenase (LDH), fructose dehydrogenase, cholin oxidase, amino oxidase and alcohol dehydrogenase.

61. The fuel cell of claim 58, wherein the enzyme carried on the cathode electrode is selected from lacase, billirubin oxidase and a complex formed of cytochrome c/cytochrome oxydase (COx).

62. The fuel cell of claim 57, wherein said HPM comprises in the non-conductive state a polymer carrying negatively charged groups that electrostatically accommodate metal cations.

63. The fuel cell of claim 62 wherein said negatively charged groups are selected from carboxyl, sulphonate and phosphate.

64. The fuel cell of claim 62 wherein said polymer is selected from polyacrylic acid, polylysine, polystyrene sulfonate and nafion.

65. The fuel cell of claim 62 wherein said metal cations are cations of transition metals.

66. The fuel cell of claim 65 wherein said metal cations are cations of metals selected from Cu, Ag, Hg, Cr, Fe, Ni and Zn.

67. The fuel cell of claim 56 having switchable conductivity properties such that in the conductive state of the HPM, the catalyst is electrically contacted with the electrode' support, thus switching on the fuel cell operation, while in the non conductive state of the HPM, the catalyst lacks electrical contact with the electrode' support, thus resulting in high electron transfer resistances switching off the fuel cell performance.

68. The fuel cell of claim 56, having tunable conductivity properties such that application of reductive potential for time-intervals that are shorter than that required for the full reduction of HPM, results in the partial reduction of the HPM to the conductive state, thus allowing tuning of the fuel cell output.

69. The fuel cell of claim 57, wherein said HPM is capable to be changed from a non-conductive to a conductive state and vice versa by reversible application of reductive potential and oxidative potential on the electrodes.

70. The fuel cell of claim 57, wherein said HPM is bound to a further polymeric layer comprising functional groups capable to bind to the catalyst layer or to the electron mediator group.

71. The fuel cell of claim 70, wherein said further polymeric layer comprises amino groups.

72. The fuel cell of claim 57, wherein the electrode' support is made of or coated by a material selected from gold, platinum, palladium, silver, carbon, copper, and indium tin oxide.

73. The fuel cell of claim 57, further comprising a membrane between the anode and the cathode.

74. The fuel cell according to claim 56 for use as a switchable and/or tunable power supply.

75. The fuel cell according to claim 56 for use as a biosensor.

76. A system for the determination of an analyte in a liquid medium comprising a biosensor according to claim 75 and a detector for measuring an electrical signal generated by said biosensor while the analyte is being oxidized or reduced, the analyte being capable of undergoing a biocatalytic oxidation or reduction in the presence of an oxidizer or reducer, respectively.

77. The system of claim 76, wherein said analyte is selected from the group consisting of sugar molecules, hydroxy, carbonyl or carboxy compounds and amino acids.

78. A system according to claim 76, wherein the biosensor is adapted for invasive measurements of an analyte in a body fluid of a tested subject.

79. A method for determining an analyte in a liquid medium, said analyte being capable to undergo a biocatalytic oxidation or reduction in the presence of an oxidizer or a reducer, respectively, the method comprising: (i) providing the system of claim 76; (ii) activating the biosensor of said system by applying reductive potential to shift the HRM of the biosensor from non-conductive into a conductive state; (iii) contacting the activated biosensor of said system with the liquid medium; (iv) measuring the electric signal generated between the cathode and the anode, said electric signal being indicative of the presence and/or the concentration of said analyte; (v) determining said analyte based on said signal.

80. The method according to claim 79, wherein said liquid medium is a body fluid, said method comprising inserting said biosensor into the body and bringing it into contact with the body fluid and determining said analyte in said body fluid within the body.

81. A method of powering an electrical device comprising the steps of electrically connecting the fuel cell of claim 56 to the device, electrooxidizing the fuel at the anode and electroreducing an electron reducing molecule at the cathode, to generate electrical power.
